(19) United States
(12) Patent Application Publication
(10) Pub. No.: US 2011/0060171 A1
(43) Pub. Date: Mar. 10, 2011

(54) Process For The Manufacture Of Pentafluorethane

(75) Inventors: Béatrice Boussand, Sainte foy Les Lyon (FR); Emmanuel Guiraud, Saint-Genis-Laval (FR)

(73) Assignee: Arkema France, Colombes (FR)

(21) Appl. No.: 12/446,161

(22) PCT Filed: Oct. 10, 2007
(86) PCT No.: PCT/FR2007/052111

§ 371 Date: Sep. 22, 2010

(30) Foreign Application Priority Data
Oct. 20, 2006 (FR) 0654390

(51) Int. Cl. C07C 17/07

(52) U.S. Cl. 570/168; 570/165; 570/169

(57) ABSTRACT

The present invention relates to a process for the manufacture of pentafluoroethane. It more particularly relates to a process for the manufacture of pentafluoroethane by gas-phase fluorination of perchloroethylene (PER) in the presence of a catalyst, characterized in that (i) the reaction of the PER with HF is carried out with an HF/PER molar ratio of greater than or equal to 20 and a pressure of greater than 5 bar absolute, and that (ii) the stream leaving this reaction step is recycled directly to the reaction step after separation of pentafluoroethane and of HCl.

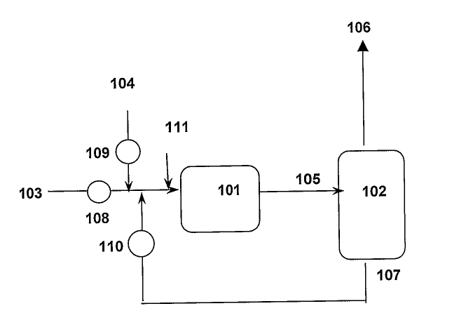

The present invention relates to a process for the manufacture of pentafluoroethane. It has more particularly as subject-matter a continuous process for the manufacture of pentafluoroethane by fluorination of perchloroethylene (PER) in the gas phase in the presence of a catalyst.

One of the critical points of a gas-phase fluorination process is the stability of the catalyst.

Several solutions have been suggested for maintaining stability of the catalyst.

Thus, the document EP 609 123 describes a continuous process for the catalytic fluorination of perchloro-ethylene in the gas phase using hydrofluoric acid in the presence of a mixed catalyst composed of oxides, halides and/or oxyhalides of nickel and of chromium deposited on a support composed of aluminum fluoride or of a mixture of aluminum fluoride and of alumina.

The stability of the catalyst is demonstrated in example 2 of this document with a temperature of 350° C., at atmospheric pressure, an HF/PER molar ratio in the vicinity of 7 and a contact time of 15 seconds.

During the fluorination of perchloroethylene using hydrofluoric acid in the presence of a catalyst, a mixture of compounds is formed with predominantly the compounds of the “F 120 series”, namely F 121 (CHCl₂—CCl₂F), F 122 (CHCl₂—CCIF₂), F 123 (CHCl₂—CF₃), F 124 (CHFCl—CF₃), F 125 (CHF₂—CF₃) or their isomers. In addition to the compounds of the “F 120 series”, the mixture comprises in particular F 115 (CF₃—CF₂Cl), F 114a (CF₃—CFCl₂), F 114 (CF₂Cl—CF₂Cl), F 133a (CH₂Cl—CF₃) and olefins F 1111 (CFCl=CCl₂) and F 1112a (CF₂=CCl₂).

Furthermore, the pressure of the fluorination reaction is often set by the separation and purification stages, in particular the separation of HCl.

Thus, the document EP 734 366 describes a process for the manufacture of pentafluoroethane by reacting, in the first stage, a perhaloethylene or pentahaloethane with HF in the gas phase in the presence of a catalyst. This document teaches the implementation of this stage at a pressure which can range up to 30 bar absolute in order to facilitate the circulation of the gas stream in the plant.

The document EP 1 110 936 describes a method for the preparation of fluoroethane compounds by reacting at least one compound chosen from PER, dichlorotrifluoro-ethane (123) and 124 with HF in the presence of a chromium oxyfluoride catalyst having a fluorine content of at least 30% by weight. This document teaches the implementation of the fluorination reaction at a pressure which will depend on the conditions for separating the products and the purification conditions.

There has now been found a process for the manufacture of pentafluoroethane by reacting perchloroethylene with HF in the gas phase in the presence of a catalyst which does not exhibit the abovementioned disadvantages.

A subject-matter of the invention is thus a process for the manufacture of pentafluoroethane from perchloro-ethylene and HF in the gas phase in the presence of a catalyst, characterized in that (i) the reaction of PER with HF is carried out with an HF/PER molar ratio of greater than or equal to 20 and a pressure greater than 5 bar absolute and that (ii) the stream exiting from this reaction stage is subjected to a separation stage in order to give a fraction (A) of light products comprising hydrochloric acid and pentafluoroethane and a fraction (B) of heavy products comprising unreacted hydrofluoric acid and possibly unreacted perchloro-ethylene and that (iii) the fraction (B) is recycled directly to the reaction stage without any purification operation.

Fraction B may additionally comprise dichlorotrifluoro-ethane and/or chlorotetrafluoroethane formed in the reaction stage.

The HF/PER molar ratio in the reaction stage is preferably between 20 and 60 and advantageously between 25 and 50.

The temperature of the reaction can be between 310 and 400° C., preferably between 330 and 375° C.

The reaction stage is preferably carried out at a pressure of between 6 and 15 bar absolute and advantageously of between 6 and 12 bar absolute.

In the process according to the present invention, the contact time divided by the absolute pressure is between 2 and 6 s/bar, preferably between 2.5 and 4 s/bar. The contact time is calculated as being the time for the gases to pass through the catalyst volume under the reaction conditions.

Any fluorination catalyst may be suitable for the process of the present invention. The catalyst used preferably comprises oxides, halides, oxyhalides or inorganic salts of chromium, aluminum, cobalt, manganese, nickel, iron or zinc, which may be supported.

Use is preferably made of a catalyst based on chromium oxide (Cr₂O₃) optionally including another metal with an oxidation state greater than zero which is selected from Ni, Co, Mn and Zn. Advantageously, this catalyst can be supported on alumina, aluminum fluoride or aluminum oxyfluoride.

For this invention, preference will be given to mixed catalysts composed of oxides, of halides and/or of oxyhalides of nickel and of chromium deposited on a support composed of aluminum fluoride or of a mixture of aluminum fluoride and of alumina, as described, for example, in patents FR 2 669 022 and EP-B-0 609 124.

When a mixed nickel/chromium catalyst is used, the catalysts comprising, by weight, from 0.5 to 20% of chromium and from 0.5 to 20% of nickel and more particularly those comprising from 2 to 10% by weight of each of the metals in a nickel/chromium atomic ratio of between 0.1 and 5, preferably in the vicinity of 1, will be recommended.

Although this is not necessary for the fluorination reaction, it may be judicious to introduce a low content of oxygen with the reactants. This content can vary, depending on the operating conditions, between 0.02 and 2 mol %, with respect to the reactants entering the reactor. The introduction of oxygen can be carried out continuously or sequentially.

The process according to the present invention can be carried out both continuously and batchwise but it is preferable to operate continuously.

### EXPERIMENTAL PART

The catalyst used is a mixed nickel/chromium catalyst with an atomic ratio Ni/Cr=1 which is supported on prefluorinated alumina and which is prepared by impregnation with solutions of nickel salt and chromic anhydride (CrO₃). After impregnating and drying, the salt is subjected to a treatment at a temperature of between 320 and 390° C. in the presence of a mixture of hydrofluoric acid and of nitrogen (concentration by volume of this acid in the nitrogen of 5 to 10%). The examples were carried out using a fluorination pilot plant composed of an evaporator, a fluorination reactor (101), a distillation column (102) and a recycling loop (107). The reactants (perchloroethylene (104) and hydrofluoric acid (103), after having evaporated them in (108) and (109)) are fed continuously in the gas phase to a fluorination reactor (101) made of Inconel comprising 48.8 l of catalyst. At the outlet of the reactor (105), a distillation column (102) makes it possible to separate the reaction products, such as F 125, HCl, HF (entrained in the azeotrope form with the organic compounds) and all or part of F 123 and F 124, on the one hand (106), from the unconverted reactants and underfluorinated products (PER, HF in excess and all or part of F 123 and F 124), on the other hand (107). The column bottom product (107), comprising the unconverted reactants and the underfluorinated products, is recycled to the reactor after having evaporated them in (110).

An unchanging level is maintained in the reboiler of the distillation column by virtue of the recycling flow rate and the flow rate of fresh reactants.

Air (111) is introduced into the reactor in an amount such that it corresponds to the molar ratio indicated below.

The operating conditions in the reactor for the fluorination of PER are as follows:
Temperature of the oven regulated at 350° C.
Pressure: 7 bar abs
HF/PER molar ratio=41
O₂/(HF+organic compounds) molar ratio=0.1%
Contact time=23.3 s (thus contact time/Pabs=3.3 s/bar)

After operating for 500 h, the productivity of F 125, recovered at the top of the distillation column, remains stable and equal to 50 g/h/1 of catalyst and the PER is completely converted at the reactor outlet. The composition (mol %) of the three main streams is stable:

| | Reactor outlet | Column top | Recycling |
|---|---|---|---|
| | (105) | (106) | (107) |
| F125 | 2.0 | 17.8 | — |
| F124/F124a | 2.4 | 1.8 | 2.5 |
| F123/F123a | 0.6 | — | 0.6 |
| F122/F122a | 0.01 | — | <0.01 |
| Others | 0.6 | 4.7 | 0.1 |
| HF | 86.2 | 3.8 | 96.8 |
| HCl | 8.2 | 71.9 | — |

### Comparative Example

Catalyst volume: 17.8 liters
Temperature of the oven regulated at 310° C.
Pressure: 7 bar abs
HF/PER molar ratio=7
O₂/(HF+organic compounds) molar ratio=0.1%
Contact time=40 s (thus contact time/Pabs=5.7 s/bar)

The results are presented in the table below:

| | PER conversion | Production of | Production |
|---|---|---|---|
| Time | at the reactor | F123 + F124 + F125 | of F125 |
| (h) | outlet (%) | (g/h/l) | (g/h/l) |
| 75 | 96.9 | 192 | 7.8 |
| 198 | 94.3 | 169 | 4.1 |
| 325 | 90.8 | 160 | 2.9 |
| 405 | 88.6 | 148 | 2 |
| 682 | 81.5 | 100 | 0.74 |
| 819 | 72.5 | 83 | 0.7 |

What is claimed is:

1. A process for the manufacture of pentafluoroethane from perchloroethylene and HF in the gas phase in the presence of a catalyst, characterized in that (i) the reaction of PER with HF is carried out with an HF/PER molar ratio of greater than or equal to 20 and a pressure greater than 5 bar absolute and that (ii) the stream exiting from this reaction stage is subjected to a separation stage in order to give a fraction (A) of light products comprising hydrochloric acid and pentafluoroethane and a fraction (B) of heavy products comprising unreacted hydrofluoric acid and possibly unreacted perchloroethylene and that (iii) the fraction (B) is recycled directly to the reaction stage without any purification operation.

2. The process as claimed in claim 1, characterized in that fraction B can additionally comprise dichlorotrifluoroethane and/or chlorotetrafluoroethane formed in the reaction stage.

3. The process as claimed in claim 1 or 2, characterized in that the HF/PER molar ratio in the reaction stage is between 20 and 60 and preferably between 25 and 50.

4. The process as claimed in any one of the preceding claims, characterized in that the reaction temperature is between 310 and 400° C., preferably between 330 and 375° C.

5. The process as claimed in any one of the preceding claims, characterized in that the reaction stage is carried out at a pressure of between 6 and 15 bar absolute and preferably of between 6 and 12 bar absolute.

6. The process as claimed in any one of the preceding claims, characterized in that the catalyst is a mixed catalyst composed of oxides, of halides and/or of oxyhalides of nickel and of chromium deposited on a support composed of aluminum fluoride or of a mixture of aluminum fluoride and of alumina.

7. The process as claimed in any one of the preceding claims, characterized in that the reaction stage is carried out in the presence of oxygen.
